(19) United States
(12) Patent Application Publication
(10) Pub. No.: US 2017/0076631 A1
(43) Pub. Date: Mar. 16, 2017

(54) RANDOMIZED WEIGHT MODULATION

(71) Applicants: Alexander Proscurshim, Boca Raton, FL (US); Igor Proscurshim, Boca Raton, FL (US)

(72) Inventors: Alexander Proscurshim, Boca Raton, FL (US); Igor Proscurshim, Boca Raton, FL (US)

(21) Appl. No.: 15/265,849

(22) Filed: Sep. 15, 2016

Related U.S. Application Data
(60) Provisional application No. 62/218,686, filed on Sep. 15, 2015.

(51) Int. Cl. G09B 19/00; A61B 5/00

(52) U.S. Cl. CPC G09B 19/0092; A61B 5/4866; A61B 5/486

(57) ABSTRACT

A method of modulating an individual's weight generates a randomized daily caloric intake value based on an individual's body composition and metabolic rates and the individual's specific weight management goals, including weight loss, weight gain, or weight control and eliminates the individual's foreknowledge of future daily caloric or nutritional intake goals. Once the individual's base metabolic rate is determined, a series of categories, each having a different daily caloric intake value, is calculated to provide a range of daily caloric intake values. A random or pseudorandom sequence of the categories is generated over a block of days during which the individual's weight is to be modulated. The category or its corresponding daily caloric intake value of a particular day is not revealed to the individual more than about 24 hours before a particular day.

### CROSS-REFERENCE TO RELATED APPLICATIONS

This application claims priority to U.S. Provisional Application Ser. No. 62/218,686 filed on Sep. 15, 2015, the contents of which are hereby incorporated in their entirety.

STATEMENT REGARDING FEDERALLY SPONSORED RESEARCH OR DEVELOPMENT

Not Applicable.

### BACKGROUND OF THE INVENTION

Field of the Invention:

The present invention relates to systems and methods for weight management by providing individualized nutritional goals. More particularly, the invention relates to improving adherence and/or efficacy of a process or method for achieving weight change or management when applied to humans or animals.

Description of the Related Art:

Obesity has become a world-wide health issue and is associated with multiple diseases such as diabetes, high blood pressure, heart disease, and cancer. In the US over 69% of the population is overweight and this number continues to increase. Abnormal weight gain is largely preventable and lifestyle adjustment remain one of the most effective methods to control an individual's weight. Not surprisingly, diet products and programs are a multimillion dollar a year industry. However, the effectiveness of these products and programs vary widely. Many individuals remain unable to find a program or weight management solution that provides effective long term results. The “yo-yo” pattern of losing and regaining weight shown in FIG. 1 is a source of frustration for most persons attempting to better manage their weight.

In the field of weight loss and weight management, existing methods rely ubiquitously on repetition of a strict and consistent regimen and predictable daily caloric and/or nutritional intake goals. Such diets may work in the near term, but are unsustainable and are often interrupted. The human body naturally seeks diversity of diet, making “cheating” on a diet inevitable. Most of these diets having a limited and/or strict regimen have a low long-term success rate. Continued adherence to any method for weight management continues to be one of the difficulties in obtaining long term weight management.

One possible reason for failure of the current approaches to weight management may lie in their predictability. For many diet programs, the individual plans the diet ahead of time and has foreknowledge of the daily caloric and/or nutritional goals from the beginning of the program or process. Predictable diet is a relatively new phenomenon for humans. Before the modern human era, most humans literally “ate what they killed or found” and ate when food was available. One day they may gorge, and the next day they may go hungry. Similarly, one day a human may languish, while another day he or she may be forced to push his or her stamina and endurance to the limit.

In view of the foregoing, there is a need to provide an improved method or system for managing weight consistently over the long term and overcomes the deficiencies described above.

It is also desirable to provide a means of effective weight management compatible with the human body and its evolution.

### BRIEF SUMMARY OF THE INVENTION

Accordingly, a primary object of the present invention is to provide an improved and maintainable weight management and dietary program. In addition, the invention provides means to avoid the repetitiveness and predictability of other methods that reduce their long term effectiveness.

A diet and activity pattern may be developed by generating unpredictable, random daily caloric or nutritional goals specific to an individual characteristics and weight management goals that are not given to the dieter until the beginning of the specific day. In one embodiment, several different daily calorie intake limits may be assigned to categories and arranged in a random pattern. An uneven distribution ratio may be applied to the categories of daily intake goals prior to arranging a sequence of categories to form a complete diet plan or a cycle of a diet plan.

Typically three to twenty categories may be randomly distributed over a number of days in a preselected distribution ratio to create a complete cycle. The cycle may be repeated, or the cycle may be changed after a given cycle is complete. The number of different daily caloric intake limits, their relative values, their distribution ratio and the length of the cycle may all be modified based on an individual's body composition, metabolic rates, and weight management goals. The invention also provides methods of preparing a sequence of daily caloric goals without informing the dieter of them until the day upon which a goal is in effect.

In one embodiment, target caloric intake goals are a multiple or fraction of the dieter's Metabolic Rate (MR) that may be, and may include one target of 0 calories, or fasting/starving. These different daily caloric intake goals, referred to herein as categories, are randomly distributed over a period of time. It is also preferable for a randomly generated distribution of the categories to avoid three consecutive days of the same category. For example, it is preferred to not have three consecutive days of fasting. An uneven distribution of categories over the time period of the diet may be preferred.

In another embodiment, the individual's specific physical information are obtained from different sources such as: data repositories (for example the apple health app, the android fit app, an electronic health record) that collect the individual's information from other applications, software or services or from connected devices that generate the individual's information such as weight scales, body composition scales etc.; from other applications, software or services; or directly from devices that generate the individual's information such as weight scales, body composition scales etc. The individual's information is processed to set up the individual's method for weight management. The processing includes: determining the individual's initial parameters; determining the parameters used to generate or select a randomized sequence of categories; determining and assigning a specific Daily Caloric Intake Goal Formula to each Category; generating or selecting a randomized sequence of Categories; assigning that sequence to specific calendar dates.

It is therefore an object of the present invention to provide a weight changing or weight management program in which a variety of daily caloric intake limits or categories may be varied according to a randomly generated pattern not generally predictable on a day by day basis.

These and other objects and advantages of the present invention will become apparent from a reading of the attached specification and appended claims. There has thus been outlined, rather broadly, the more important features of the invention in order that the detailed description thereof that follows may be better understood, and in order that the present contribution to the art may be better appreciated. There are features of the invention that will be described hereinafter and which will form the subject matter of the claims appended hereto.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a graph of an obese individual's weight fluctuation with different weight management methods in the period between December 2013 and July 2015;

FIG. 2 is a flow-chart showing steps involved in the initial set-up of an exemplary embodiment of the method with the input of exemplary information and processing of that information for use in subsequent steps in accordance with the principles of the invention;

FIG. 3 is a flow-chart showing steps involved in the generation of a calendar with randomized caloric intake goals for each day of an exemplary embodiment of the method using the information collected and generated from the exemplary method in FIG. 2 in accordance with the principles of the invention;

FIG. 4 is a flow-chart showing steps of an exemplary embodiment of the method involved in the daily input and processing of the individual's information in accordance with the principles of the invention;

FIG. 5 is a home screen of a user interface for a software application for a mobile device in accordance with the principles of the invention;

FIG. 6 is a graph showing the change in weight in lbs. over 30 days of Subjects 001 and 004 of Example 1 in accordance with the principles of the invention;

FIG. 7 is a graph showing the change in weight in lbs. over 30 days of the Subjects 002 and 003 of Example 1 in accordance with the principles of the invention;

FIG. 8 is a graph showing the change in BMI for all four Subjects of Example 1 in accordance with the principles of the invention;

FIG. 9 is a graph showing the cumulative weight loss in lbs. of all four Subjects of Example 1 in accordance with the principles of the invention.

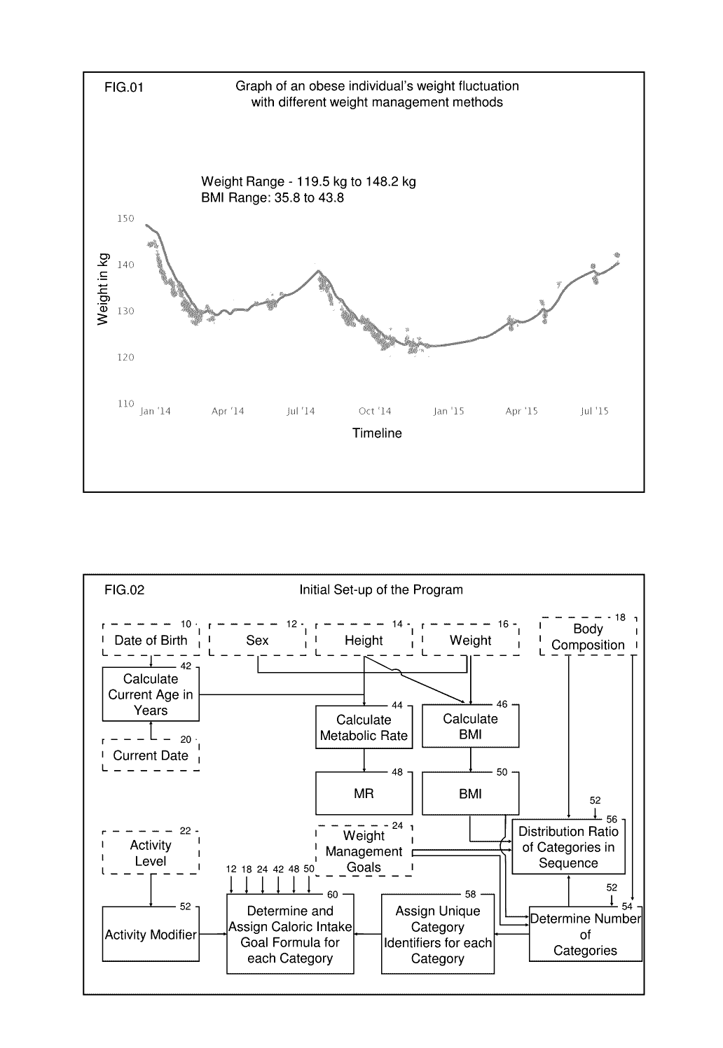

### DETAILED DESCRIPTION

The invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The disclosed subject matter is described with reference to the drawings, wherein like reference numerals are used to refer to like elements throughout. In the following description, for purposes of explanation, numerous specific details are set forth in order to provide a thorough understanding of the various embodiments of the subject disclosure. It may be evident, however, that the disclosed subject matter may be practiced without these specific details. In other instances, well-known structures and devices are shown in block diagram form in order to facilitate describing the various embodiments herein.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

Some of the principles of the invention arise from the fields of nutrition, health and mathematics. Therefore, there are a number of terms used throughout this application that may be considered terms of arts in one or more of these technical fields. One of ordinary skill in the art would recognize the particular meanings of these terms specific to the relevant discipline.

Disclosed is an invention providing a method that generates randomized daily caloric or nutritional intake goals based on an individual's body composition and metabolic rates; the individual's specific weight management goals, including weight loss, weight gain, or weight control; and eliminates the individual's foreknowledge of future daily caloric or nutritional intake goals. The method will lead to an improved and maintainable weight management and dietary pattern. The method avoids the repetitiveness and predictability of other methods by generating a random daily caloric or nutritional goal based on the individual's characteristics and the individual's weight management goals, and avoids the prescience or foreknowledge by informing the nutritional goal for a specific day only on that specific day.

The methods described herein may provide a system of weight management that may be used to maintain a constant weight, to decrease weight and/or to increase weight. The two main aspects of the method are: 1) the randomization of different and unique pre-defined categories to a future schedule of hours, days, or weeks, and 2) the elimination of the foreknowledge of the future schedule of categories. In most embodiments the individual or animal will be blinded to the future sequence of categories and will not know what the next scheduled category is. However, in certain embodiments of the invention the individual or animal may have some foreknowledge of the category sequence or methods for generating a calendar. These aspects can be applied jointly or separately to improve the ability of an individual to manage his or her weight, to increase adherence, and/or increase efficacy.

One exemplary embodiment of a weight modulation method in accordance with the principles of the invention begins by determining a base metabolic rate for an individual. The base metabolic rate is then used to calculate a plurality of different daily caloric intake values, referred to as categories. These daily caloric intake values for the categories is calculated based upon the amount of weight an individual may wish to lose or gain, the speed at which an individual desires his or her weight to change, and the amount of variants an individual desires between maximum and minimum daily caloric intake values. Next, these categories may be placed in a random sequence using a random number generator, or RNG, where each category is assigned a number and inserted into the sequence. The block of time encompassed by a sequence is generally at least seven or more days, but may be one month, one year or another desired time interval. Optionally, a block of time may be assigned a sequence consisting of more than one sequence added together.

The sequence for the block of days over which an individual's weight is to be modulated may be purely random. It may be desirable to create the sequence of the categories using a pseudo random number generator, or PNRG, instead of a purely random sequence. Mid-range daily caloric intake values may be weighted more heavily. Conversely the high and low ends of the daily caloric intake values may be weighted. Those skilled in the art will appreciate that there are numerous methods of applying probability product densities over a range of discrete values in order to provide a sequence that is not entirely and purely random. This may be desirable to avoid fluctuations between subsequent daily intake values that cause undue stress on a particular individual. Using probability density mathematics to modify a random sequence is a well-known method of operating upon a randomized sequence of discrete values. It may also be desirable to further modify the sequence generator to avoid repetition of values within a sequence. For example, an individual desiring to lose a substantial amount of weight rapidly may choose to include a category in which the daily caloric intake value is zero. However, it is in general undesirable to have a sequence of 2, 3, 4 or more days in a row of zero caloric intake. Similarly, it would be undesirable to have several days in a row of ingesting an unusually high number of calories. Further, without being bound by theory, the inventors believe that by alternating daily caloric intake, the body's natural systems for controlling average weight become “confused” in a manner similar to the way bodybuilders believe that alternating exercise styles “confuses” muscles in order to stimulate a better response. Therefore, it would be undesirable to also have a series of several days in a row of the same daily caloric intake for any particular category, because this would defeat the purpose of using a randomized daily caloric intake method in the first place.

### 1. Obtaining Preliminary Information Required to Prepare a Weight Modulation Method

First, an individual's specific physical information may be inputted by the individual and/or collected from various sources such as pre-existing databases, e.g. smartphone or mobile health applications or an electronic health record. Optionally, the information may be obtained from other applications, software or services or from connected devices that generate the individual's information such as weight scales, body composition scales etc. or may be inputted manually. The individual's information is processed to set up the individual's method for weight management. The processing may include determining the parameters used to generate or select a randomized sequence of categories, determining and assigning a specific Daily Caloric Intake Goal Formula to each Category, generating or selecting a randomized sequence of Categories, and assigning that sequence to specific calendar dates. Once the general, physical data of a particular individual is obtained, a dieter's, Metabolic Rate(MR) and Body Mass Index (BMI) may be calculated. This allows the dieter or weight manager to determine a base metabolism to which the Daily Caloric Intake Goal Formula for that specific day is applied to give the base daily caloric/nutritional intake.

In addition to information regarding the purely physical characteristics of a person, additional data is gathered including the desired weight that a person wishes to achieve and the speed at which the person desires to lose weight.

FIG. 2 shows the initial steps of a system for weight management. In this initial set up, data relevant to weight management is collected. This includes collecting information shown in the boxes outlined in dashed lines and includes the date of birth 10, sex 12, height 14, weight 16, body composition 18, the current date 20, activity level 22, and weight management goals 24.

### 2. Calculating BMR

Once the basic information is known for a dieter, the number and nature of the Categories used to create the diet plan may be selected. This can include calculation of the dieter's age 42 using the age 10 and current date 20. The dieter's MR 48 and BMI 50 may be calculated in steps 44 and 46 using the dieter's age 42, sex 12, height 14 and weight 16. There are several methods for calculating basal metabolic rate (BMR) of an individual using some or all of the information from steps 10, 12, 14, 16, 18, 20 and/or 42.

BMR is strictly defined as the minimal rate of energy expenditure per unit time by endothermic animals at rest. It is reported in energy units per unit time ranging from watt (joule/second) to ml O2/min or joule per hour per kg body mass J/(h·kg)). Proper measurement requires a strict set of criteria be met. These criteria include being in a physically and psychologically undisturbed state, in a thermally neutral environment, while in the post-absorptive state (i.e., not actively digesting food). In bradymetabolic animals, such as fish and reptiles, the equivalent term standard metabolic rate (SMR) is used. It follows the same criteria as BMR, but requires the documentation of the temperature at which the metabolic rate was measured. This makes BMR a variant of standard metabolic rate measurement that excludes the temperature data, a practice that has led to problems in defining “standard” rates of metabolism for many mammals.

Metabolism refers to the processes that the body needs to function. Basal Metabolic Rate is the amount of energy expressed in calories that a person needs to keep the body functioning at rest. Some of those processes are breathing, blood circulation, controlling body temperature, cell growth, brain and nerve function, and contraction of muscles. Basal metabolic rate (BMR) affects the rate that a person burns calories and ultimately whether that individual maintains, gains, or loses weight. The basal metabolic rate accounts for about 60 to 75% of the daily calorie expenditure by individuals. It is influenced by several factors. BMR typically declines by 1-2% per decade after age 20, mostly due to loss of fat-free mass, although the variability between individuals is high.

Precise calculation of a BMR of a human being or any mammal can be an extremely extensive and complex task. As a practical matter, a person's BMR is generally estimated using one of the standard accepted methods for approximating the BMR using a person's weight, height, age, sex and other easily ascertainable observable scaler quantities. Some of the more commonly used methods include the Harris-Benedict Equation, the Revised Harris-Benedict Equation, the Mifflin St Jeor Equation, the Katch-Mcardle Formula and the Institute of Medicine Formula.

The dieter's activity level 22 may be used to calculate Activity Modifiers 52. It is therefore not uncommon to adjust and individuals estimated BMR (also sometimes referred to as simply “MR”) to account for his or her general level of activity. These adjustments are typically referred to as Activity Modifiers. If a person is particularly physically active, his or her BMR IS PROBABLY HIGHER THAN THAT ESTIMATED BY ONE OF THE ABOVE METHODS. A weight management plan's Activity Modifiers may be determined based upon a dieter's activity level 22 and MR 48. The Activity Modifier may be included in Daily Caloric Intake Goal Formula for a Category. Table 2 shows an exemplary group of Activity Modifiers proposed for use with the Harris-Benedict Equation.

TABLE 2

| Activity Level | Modifier |
|---|---|
| Little to no exercise | MR × 1.2 |
| Light exercise (1-3 days per week) | MR × 1.375 |
| Moderate exercise (3-5 days per week) | MR × 1.55 |
| Heavy exercise (6-7 days per week) | MR × 1.725 |
| Very heavy exercise (twice per day, extra heavy) workouts) | MR × 1.9 |

### 3. Generating a Daily Caloric Intake Calendar

A daily caloric intake calendar in accordance with the principles of the invention is created by selecting a domain of discrete daily caloric intake values, referred to herein as categories, and arranging them in a chronological sequence, usually using a pseudorandom number generator to assign categories to particular days. The dieter's body composition 18, weight management goals 24, BMI 50, and the selected Activity Modifiers 52 may be used to determine the number of Categories to be used. After determining the number of Categories, a distribution ratio 56 for the Categories may be selected. For convenience, Category Identifies may be selected for each Category in step 58.

A Daily Caloric Intake Goal Formula is determined 60 for each Category 58 based on the individual's sex 12, body composition 18, weight management goals 24, age 42, metabolic rate 48, and BMI 50. An exemplary formula for Daily Caloric Intake Goal Formula for a Category could be: Daily Caloric Intake Goal=x·MR+MR·(Activity Modifier−1)+Adjustment 1+Adjustment 2 +. . . Adjustment n, where x is a specific percentage selected from the range from 0 to 600% depending on the individual's information 12, 18, 42, 50, 52 and weight management goal 24, where current BMR will be calculated for that specific date using the individual's information for the specific date, where the Activity Modifier is determined by the user 52, and where Adjustments 1 through n are determined based on the individual's information 12, 18, 24, 42, 50, 52, as well as using other parameters. A Daily Caloric Intake Goal Formula for a Category may have any or all of the components exemplified above and may have different values of x. Each Category should have a Daily Caloric Intake Goal Formula assigned to it.

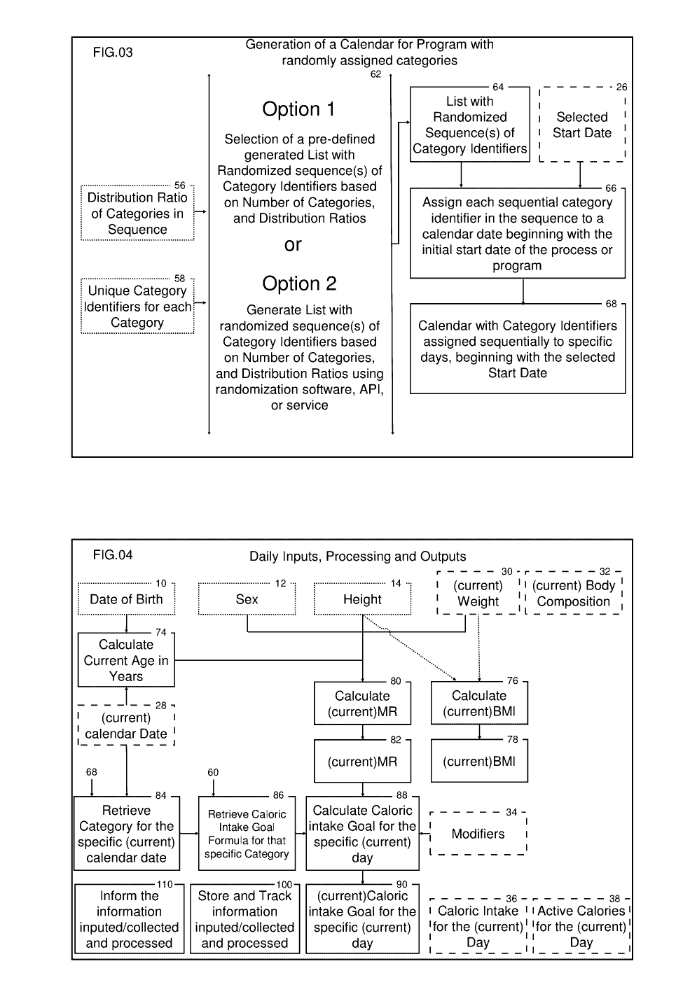

FIG. 3 shows two Options 62 for generating a weight management plan or schedule for a specific number of days, referred to herein as a block, where the number of days is greater than the number of categories. The Distribution Ratio 56 and Category Identifiers 58 may be used to select a predefined and previously generated list, generated using software and algorithms, with a randomized sequence of Categories Identifiers based upon the number of Categories and Distribution Ratio where each category identifier on the sequence represents a category that will be assigned to a specific date. Alternatively, the Distribution Ratio and the Category Identifiers may be used to generate a sequence of randomized Categories using software and algorithms where each category identifier on the sequence represents a category that will be assigned to a specific date. The random sequence of Category Identifiers are then assigned to specific calendar dates starting with the selected start date 26 to generate the weight management plan or schedule 68. The Categories are randomly distributed over a period of time such that their relative frequencies comport to the selected distribution ratio. It may be preferable that a randomly generated distribution of Categories avoid two or three consecutive days of the same category. For example, it is preferred to not have two or three consecutive days of fasting. An uneven distribution of categories over the time period of the diet may be preferred. Those skilled in the art will appreciate that weighing different components of a domain and avoiding repetition are common methods of adjusting randomizing functions.

FIG. 4 shows how the weight management plan selects a specific caloric intake goal 84 based upon the actual current date 28 and retrieves 86 the Daily Caloric Intake Goal Formula 60 for the Category 68 assigned to that specific date. The caloric intake goal for that specific date may then be calculated 88 using the individual's current Metabolic Rate which is calculated on the specific date using the individual's age 74, sex 12, height 14, and current weight 30, activity modifier 52, and other modifiers 34. The individual's information, progress, and changing weight 30, body composition 32, BMI 78, and MR 82 may be recorded or stored for later analysis or tracking. Target caloric intake goals, are calculated on and for each specific date by using the Daily Caloric Intake Goal Formula that corresponds to the Category assigned to that specific date. The individual's current Metabolic Rate (MR), Activity Modifiers and other Modifiers are variables that are inputted into that specific date's Daily Caloric Intake Goal Formula to calculate the corresponding caloric intake goal. The result of the formula calculation may be 0 which corresponds to a day of fasting.

The outcome of the use of the exemplified method depends primarily on the individual's weight-management goal and on the individual's adherence to the caloric intake goals. If the individual's weight management goal is to lose weight the method will result in a cumulative caloric loss, when subtracting the expected daily expenditure from the daily caloric intake goal over the period in which the method is being used. The more aggressive the individual's selection of weight-loss programming, the greater the cumulative deficit will be. The actual amount of weight loss for the method will depend on the individual's adherence to maintaining the actual caloric intake below the caloric intake goal. If the individual's weight management goal is to gain weight the method will result in a cumulative gain, when subtracting the expected daily expenditure from the daily caloric intake goal, over the period in which the method is being used. The actual amount of weight gain for the method will depend on the individual's adherence to maintaining the actual caloric intake within the caloric intake goal. If the individual's weight management goal is to maintain weight, the method will result in a cumulative difference close to 0, when subtracting the expected daily expenditure from the daily caloric intake goal, over the period in which the method is being used. The actual amount of weight gain or loss for the method will depend on the individual's adherence to maintaining the actual caloric intake below the caloric intake goal.

The random sequence of pre-defined categories can be generated manually, using either a spreadsheet software formula; a statistical software package such as R-package, SAS, MATLAB, or other statistical software; a randomization Application Program Interface; an open source or proprietary randomization software; using randomization services offered by people or companies; a randomization device; or any other form of generating randomized sequences of pre-defined categories. The list or sequence can be of any size greater than the number of pre-defined categories and can be generated using a single block of randomly assigned or distributed categories, using multiple blocks of sequences of randomly assigned or distributed categories where blocks can be of the same length or size or of different lengths or sizes; using permuted block randomization; using simple randomization, or any other randomization method that generates a randomized sequence of a set of pre-defined categories or randomly assigns or allocates a set of predefined categories to a sequence. In certain embodiments, randomization can maintain a fixed distribution ratio for each category to generate a sequence with a specific pre-defined ratio of the pre-defined categories. These sequences can be of any size greater than the number of pre-defined categories. A block or sequence may be further modified, discarded or selected based on criteria such as to exclude sequential repeats of specific categories, a specific sequence of categories, or other criteria whose objective is to increase adherence to the method.

For example, Table 3 shows a sequence of 120 categories generated from the random allocation of 5 unique categories using the Category Identifiers: A,B,C,D,Z with an uneven distribution of 1:3:3:2:1, respectively, in blocks of 10. Those skilled in the art will appreciate that this distribution ratio represents a probability distribution for a domain of finite, discrete components.

TABLE 3

| | Sequence | |
|---|---|---|
| Sequence | within | |
| ID | Block | Category |
| 1 | 1 | D |
| 2 | 2 | B |
| 3 | 3 | D |
| 4 | 4 | C |
| 5 | 5 | C |
| 6 | 6 | A |
| 7 | 7 | Z |
| 8 | 8 | C |
| 9 | 9 | B |
| 10 | 10 | B |
| 11 | 1 | A |
| 12 | 2 | Z |
| 13 | 3 | D |
| 14 | 4 | B |
| 15 | 5 | C |
| 16 | 6 | B |
| 17 | 7 | D |
| 18 | 8 | B |
| 19 | 9 | C |
| 20 | 10 | C |
| 21 | 1 | C |
| 22 | 2 | D |
| 23 | 3 | B |
| 24 | 4 | C |
| 25 | 5 | C |
| 26 | 6 | D |
| 27 | 7 | B |
| 28 | 8 | B |
| 29 | 9 | A |
| 30 | 10 | Z |
| 31 | 1 | B |
| 32 | 2 | C |
| 33 | 3 | B |
| 34 | 4 | Z |
| 35 | 5 | C |
| 36 | 6 | C |
| 37 | 7 | D |
| 38 | 8 | D |
| 39 | 9 | A |
| 40 | 10 | B |
| 41 | 1 | C |
| 42 | 2 | C |
| 43 | 3 | B |
| 44 | 4 | Z |
| 45 | 5 | A |
| 46 | 6 | B |
| 47 | 7 | D |
| 48 | 8 | D |
| 49 | 9 | B |
| 50 | 10 | C |
| 51 | 1 | D |
| 52 | 2 | B |
| 53 | 3 | C |
| 54 | 4 | B |
| 55 | 5 | C |
| 56 | 6 | A |
| 57 | 7 | Z |
| 58 | 8 | C |
| 59 | 9 | B |
| 60 | 10 | D |
| 61 | 1 | B |
| 62 | 2 | C |
| 63 | 3 | D |
| 64 | 4 | C |
| 65 | 5 | B |
| 66 | 6 | C |
| 67 | 7 | D |
| 68 | 8 | Z |
| 69 | 9 | B |
| 70 | 10 | A |
| 71 | 1 | B |
| 72 | 2 | C |
| 73 | 3 | B |
| 74 | 4 | B |
| 75 | 5 | D |
| 76 | 6 | D |
| 77 | 7 | A |
| 78 | 8 | C |
| 79 | 9 | Z |
| 80 | 10 | C |
| 81 | 1 | D |
| 82 | 2 | A |
| 83 | 3 | B |
| 84 | 4 | B |
| 85 | 5 | D |
| 86 | 6 | C |
| 87 | 7 | C |
| 88 | 8 | Z |
| 89 | 9 | B |
| 90 | 10 | C |
| 91 | 1 | Z |
| 92 | 2 | D |
| 93 | 3 | B |
| 94 | 4 | A |
| 95 | 5 | B |
| 96 | 6 | B |
| 97 | 7 | C |
| 98 | 8 | C |
| 99 | 9 | D |
| 100 | 10 | C |
| 101 | 1 | C |
| 102 | 2 | C |
| 103 | 3 | D |
| 104 | 4 | Z |
| 105 | 5 | B |
| 106 | 6 | B |
| 107 | 7 | D |
| 108 | 8 | A |
| 109 | 9 | B |
| 110 | 10 | C |
| 111 | 1 | C |
| 112 | 2 | D |
| 113 | 3 | Z |
| 114 | 4 | C |
| 115 | 5 | A |
| 116 | 6 | B |
| 117 | 7 | C |
| 118 | 8 | B |
| 119 | 9 | D |
| 120 | 10 | B |

For the example in Table 3, on day 1, the category identifier for caloric goal calculation is: D; on day 92 the category for caloric goal calculation is A and so on. In one embodiment, These formulas assigned to each of these category identifiers can be defined as follows for aggressive weight loss:

A: Caloric Intake Goal=0%MR

B: Caloric Intake Goal=50%MR

C: Caloric Intake Goal=75%MR

D: Caloric Intake Goal=100%MR

Z: Caloric Intake Goal=100%MR +MR·(Activity Modifier−1)

Table 4 shows the sequence of categories of the list exemplified in Table 3 assigned to a specific calendar date beginning on Jul. 27, 2015.

TABLE 4

| Calendar Date | Category |
|---|---|
| Jul. 27, 2015 | D |
| Jul. 28, 2015 | B |
| Jul. 29, 2015 | D |
| Jul. 30, 2015 | C |
| Jul. 31, 2015 | C |
| Aug. 1, 2015 | A |
| Aug. 2, 2015 | Z |
| Aug. 3, 2015 | C |
| Aug. 4, 2015 | B |
| Aug. 5, 2015 | B |
| Aug. 6, 2015 | A |
| Aug. 7, 2015 | Z |
| Aug. 8, 2015 | D |
| Aug. 9, 2015 | B |
| Aug. 10, 2015 | C |
| Aug. 11, 2015 | B |
| Aug. 12, 2015 | D |
| Aug. 13, 2015 | B |
| Aug. 14, 2015 | C |
| Aug. 15, 2015 | C |
| Aug. 16, 2015 | C |
| Aug. 17, 2015 | D |
| Aug. 18, 2015 | B |
| Aug. 19, 2015 | C |
| Aug. 20, 2015 | C |
| Aug. 21, 2015 | D |
| Aug. 22, 2015 | B |
| Aug. 23, 2015 | B |
| Aug. 24, 2015 | A |
| Aug. 25, 2015 | Z |
| Aug. 26, 2015 | B |
| Aug. 27, 2015 | C |
| Aug. 28, 2015 | B |
| Aug. 29, 2015 | Z |
| Aug. 30, 2015 | C |
| Aug. 31, 2015 | C |
| Sep. 1, 2015 | D |
| Sep. 2, 2015 | D |
| Sep. 3, 2015 | A |
| Sep. 4, 2015 | B |
| Sep. 5, 2015 | C |
| Sep. 6, 2015 | C |
| Sep. 7, 2015 | B |
| Sep. 8, 2015 | Z |
| Sep. 9, 2015 | A |
| Sep. 10, 2015 | B |
| Sep. 11, 2015 | D |
| Sep. 12, 2015 | D |
| Sep. 13, 2015 | B |
| Sep. 14, 2015 | C |
| Sep. 15, 2015 | D |
| Sep. 16, 2015 | B |
| Sep. 17, 2015 | C |
| Sep. 18, 2015 | B |
| Sep. 19, 2015 | C |
| Sep. 20, 2015 | A |
| Sep. 21, 2015 | Z |
| Sep. 22, 2015 | C |
| Sep. 23, 2015 | B |
| Sep. 24, 2015 | D |
| Sep. 25, 2015 | B |
| Sep. 26, 2015 | C |
| Sep. 27, 2015 | D |
| Sep. 28, 2015 | C |
| Sep. 29, 2015 | B |
| Sep. 30, 2015 | C |
| Oct. 1, 2015 | D |
| Oct. 2, 2015 | Z |
| Oct. 3, 2015 | B |
| Oct. 4, 2015 | A |
| Oct. 5, 2015 | B |
| Oct. 6, 2015 | C |
| Oct. 7, 2015 | B |
| Oct. 8, 2015 | B |
| Oct. 9, 2015 | D |
| Oct. 10, 2015 | D |
| Oct. 11, 2015 | A |
| Oct. 12, 2015 | C |
| Oct. 13, 2015 | Z |
| Oct. 14, 2015 | C |
| Oct. 15, 2015 | D |
| Oct. 16, 2015 | A |
| Oct. 17, 2015 | B |
| Oct. 18, 2015 | B |
| Oct. 19, 2015 | D |
| Oct. 20, 2015 | C |
| Oct. 21, 2015 | C |
| Oct. 22, 2015 | Z |
| Oct. 23, 2015 | B |
| Oct. 24, 2015 | C |
| Oct. 25, 2015 | Z |
| Oct. 26, 2015 | D |
| Oct. 27, 2015 | B |
| Oct. 28, 2015 | A |
| Oct. 29, 2015 | B |
| Oct. 30, 2015 | B |
| Oct. 31, 2015 | C |
| Nov. 1, 2015 | C |
| Nov. 2, 2015 | D |
| Nov. 3, 2015 | C |
| Nov. 4, 2015 | C |
| Nov. 5, 2015 | C |
| Nov. 6, 2015 | D |
| Nov. 7, 2015 | Z |
| Nov. 8, 2015 | B |
| Nov. 9, 2015 | B |
| Nov. 10, 2015 | D |
| Nov. 11, 2015 | A |
| Nov. 12, 2015 | B |
| Nov. 13, 2015 | C |
| Nov. 14, 2015 | C |
| Nov. 15, 2015 | D |
| Nov. 16, 2015 | Z |
| Nov. 17, 2015 | C |
| Nov. 18, 2015 | A |
| Nov. 19, 2015 | B |
| Nov. 20, 2015 | C |
| Nov. 21, 2015 | B |
| Nov. 22, 2015 | D |
| Nov. 23, 2015 | B |

To further exemplify the following scenario was used for the example in Table 3: the individual is a man born on Mar. 8 1983, 32 years of age and on day 1, July 27 weighs 134 kgs, is 185 cm tall, inputs a sedentary basal activity modifier (1.2), and selects an aggressive weight loss goal. The individual's MR for day 1 using the Mifflin St Jeor equation [MR=10*weight(kg)+6.25*height(cm)−5*age(y)+5(man)] for day 1 is 2341 kcals. Using the list in Table 4 the individual's caloric intake goal for July 27 would be calculated using the formula for category D: Caloric Intake Goal=100%MR=100%2341=2341 calories. For August 29 the individual inputs a weight of 128 kg, his MR for that day is 2281 kcals and the caloric intake goal would be calculated using formula for category B: Caloric Intake Goal=50%MR=50%2281=1141 calories (rounded up to nearest calorie). For September 8, the individual inputs a weight of 126 kg, his MR for that day is 2261 kcals and the caloric intake goal would be calculated using the formula for category Z: Caloric Intake Goal=100%MR+MR·(Activity Modifier−1)=100%2261+2261(1.2−1)=2713 calories (rounded to nearest calorie). For September 20, the individual inputs a weight of 124kg, his MR for that day is 2241 kcals and the caloric intake goal would be calculated using the formula for category A: Caloric Intake Goal=0%MR=0%2241=0 calories (or fasting).

In one embodiment, the individual is only informed of the daily caloric or nutritional intake goal on the beginning of that specific day. The most recent weight and height as well as the individual's activity modifier, weight management goal and that day's category (from the list generated at the start of the program) are used to calculate that day's caloric or nutritional intake goal. The individual's caloric or nutritional intake for the specific day, which may be recorded manually in a journal or using an application or software will be collected to track the individual's daily achievement. In certain embodiments the information regarding the categories or levels of caloric or nutritional intake may be informed prior to the specific day.

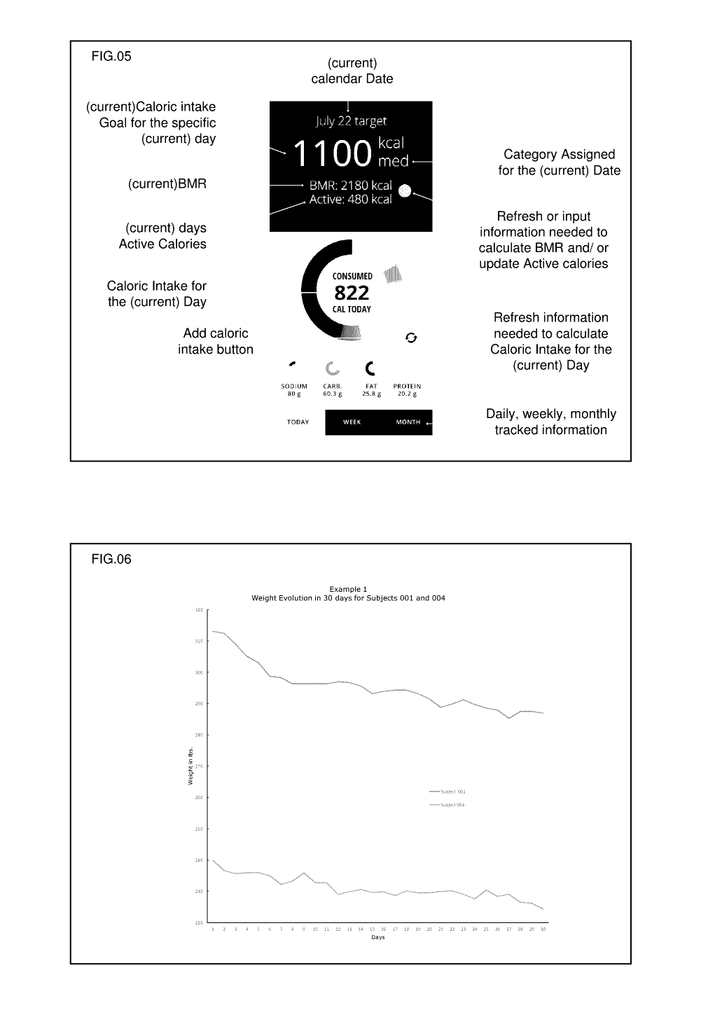

Furthermore the invention may be used to create a software, application or device that collects the individual's information, processes that information generating caloric or nutritional intake categories randomly assigned to calendar dates, establishes caloric or nutritional intake formulas for each category, collects the individual's current information to calculate the daily caloric or nutritional intake goal for that date using the caloric or nutritional intake formula associated with the category assigned to that date, collects the individual's daily caloric or nutritional intake information, stores and tracks and presents the information. The home screen of an exemplary software application for mobile devices is depicted in FIG. 5.

One of the important aspects of the present invention is the use of a PNRG applied to a set of discrete daily caloric intake values, each representing a category. Without being bound by theory, the inventors believe that the unpredictability of each day's caloric intake prevents an individual's body from compensating for a change in an individual's diet when the change is consistent and predictable. The inventors believe that this prevents the body from resisting a change in body weight. However, many aspects of specific applications of a weight plan in accordance with the invention, such as calculating an accurate BMR for an individual, determining an optimum number of categories, selecting an optimal probability ratio and selecting an optimal block of time for a generated sequence are generally not susceptible to simple and accurate calculation. The parameters and factors affecting these various aspects of the system are varied, overabundant and probably not all entirely properly identified. Therefore as a practical matter, an individual may select a particular set of categories, a probability ratio and a block of time over which to generate a pseudorandom sequence with which to begin a diet plan. If, after some period of time, such as two weeks, an individual is not satisfied with the results or the difficulty in complying with the caloric intake demands of the plan, he or she may opt to adjust some of the parameters and create a modified plan. For example, an individual may select different or additional categories of daily caloric intake value. Optionally, the individual may apply a different distribution ratio to the categories. An individual may also adjust the time period over which a sequence of categories is calculated. Optionally, if an individual is pleased with a system and sequence he or she has been using, he or she may simply repeat the sequence once completed.

In order to enhance the randomness and unpredictability of the invention, it is generally desirable for an individual to not have advanced warning of a following days daily caloric intake. Therefore, it is generally preferable for an individual's plan to be calculated and a sequence determines by a different individual or by using a program to automatically generate a sequence of categories. Such a program can be hosted on an Internet server, be Incorporated into a desktop or laptop computer or even be created as a smart phone application. The other individual or the software program keeps future and upcoming categories hidden from the individual on the plan. Typically, an individual learns of a particular days caloric intake the morning of that day or the evening before. Of course, an individual can always generate a pseudorandom sequence and never view more than the upcoming day, but many individuals may not wish to have the temptation.

Individuals may not always strictly adhere to a particular days calorie intake restrictions. Or, a person's body may change at a rate different than expected. One option that may be advantageous for use with the present invention is re-calculating an individual's BMR every day and using it to recalculate the actual value of a category for a particular day. Thus, a weight loss plan in accordance with the principles of the invention may be self adjusting from day to day in order to compensate for non-adherents to daily caloric intake restrictions and other more nebulous factors that are difficult to quantify and qualify.

Furthermore the invention may be used to create a software, application or device that: collects information relevant to the desired effect; processes that information; establishes categories with associated with processes, actions, and/or formulas to achieve the desired effect based on the information; generates a schedule of randomly assigned categories; collects information relevant to the desired effect for the current scheduled category; collects and updates information relevant to the desired effect; stores and tracks and presents the information.

FIG. 5 shows an exemplary user interface for a smartphone application that may be used for performing the randomized weight management plan of the invention.

### EXAMPLE I

### Aggressive Weight Loss with Aggressive Caloric Restriction used by 4 Subjects

Calculation of MR: The Mifflin St. Jeor MR equation may be used, as it reasonably estimates the metabolic rate without added activity:

Men: MR=10* weight(kg)+6.25*height(cm)−5*age(y)+5

Women: MR=10* weight(kg)+6.25*height(cm)−5*age(y)−161

Categories: Five categories were calculated as follows:

A=0% MR (fasting) with the instruction that if one feels lightheaded or a headache on should consume a fruit or a protein bar

B=50% MR

C=75% MR

D=100% MR

Z=100%MR+activity modifier (using the Harris Benedict modifier of 1.2*MR for sedentary)=100%MR+MR·(1.2−1)−the equivalent of a net zero day

Random Distribution of Categories in a preselected ratio: 2 random sequence lists were generated with the following characteristics: Block size 10 days, 5 Categories A, B, C, D, Z, with an uneven distribution ratio of 1:3:3:2:1 (ABBBCCCDDZ).

Equipment used by each subject: scale, smartphone with calorie counting app MyfitnessPal.

Information used for each participant at start: date of birth (to generate Age); sex; weight; height.

Instructions for weighing: Use same scale, weigh self preferably every morning after using the bathroom either naked or with underwear, taking the care to always weigh self in the same manner.

To eliminate foreknowledge of upcoming categories, two lists were generated and the initial participants divided into 2 groups. Each group knows only the others' list and each morning (prior to 6 am) one group informs the other of that day's category and corresponding target via SMS or email. This step can be replaced using a phone app or software.

Every day the participants weigh themselves (using a home scale, Withings Body Analyzer or Weight-Watchers Scale) in the morning, calculate their Caloric Intake Goal using the Caloric Intake goal Formula for that day's Category, record their caloric intake using the MyFitness Pal App, and restrict their intake to that day's Goal.

Participants for the first 30-day cycle:

Group A−Assigned List 101736037476044

Subject 004 (29 yo man, Initial weight: 239.9 lbs., BMI: 32.99)

Subject 003 (31 yo woman, Initial weight: 195.1 lbs., BMI: 28.9)

Group B−Assigned List 101736037376041

Subject 002 (32 yo woman; Initial weight: 149.7 lbs.; BMI: 28.27)

Subject 001 (38 yo man; Initial weight 313.1 lbs.; BMI: 41.5)

The data (weight and caloric intake) was logged by each participant and informed to the Principal Investigator (Igor Proscurshim) at the end of every 10 day cycle.

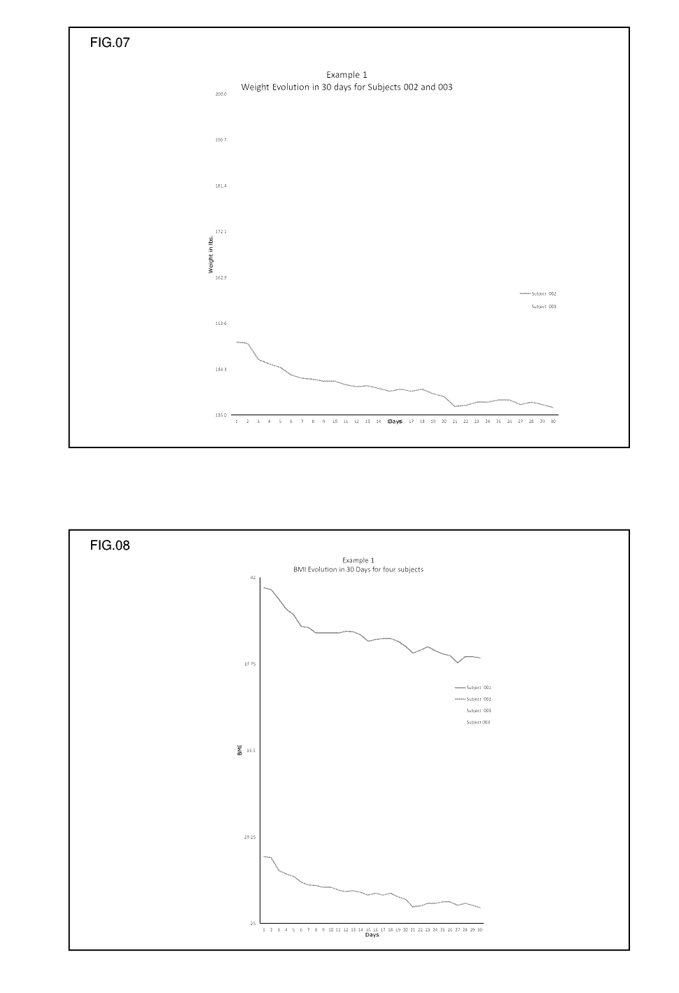

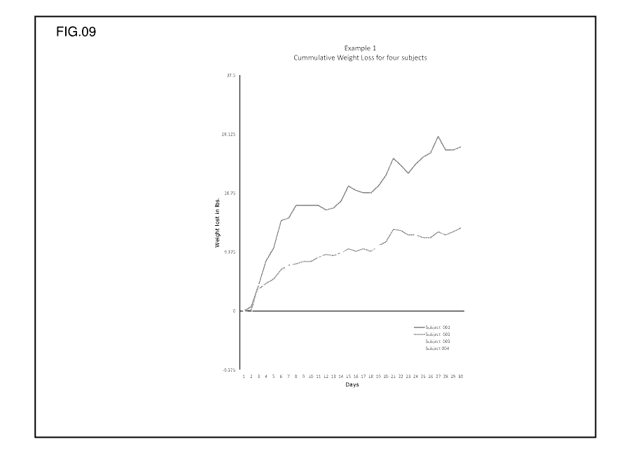

FIG. 6 shows the change in weight in lbs. of Subjects 001 and 004. FIG. 7 shows the change in weight in lbs. of Subjects 002 and 003. FIG. 8 shows the change in BMI of all four Subjects. FIG. 9 shows the cumulative weight loss in lbs. of all four Subjects. As may be seen, there is a correlation between the BMI and the change in weight. This indicates that the weight loss is primarily loss of fat tissue, and is not primarily due to decreased muscle or uniform loss of all types of tissue. As may be seen from these graphs, all four Subjects experienced a substantial change in their weight over the 30 day period.

Whereas, the present invention has been described in relation to the drawings attached hereto, it should be understood that other and further modifications, apart from those shown or suggested herein, may be made within the spirit and scope of this invention. Descriptions of the embodiments shown in the drawings should not be construed as limiting or defining the ordinary and plain meanings of the terms of the claims unless such is explicitly indicated.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

1. A method of modulating an individual's body weight comprising:
using the individual's information and weight management preferences to generate three or more categories whereas each category comprises a formula for a daily caloric intake goal;
selecting a random uneven distribution ratio to be applied to generate a random sequence of these categories;
forming a diet schedule by randomly assigning categories to days of a predetermined cycle length such that they occur in the selected uneven distribution ratio;
advising the individual of the category of a given day only on the given day itself such that the individual does not know the categories of future days of the diet plan; and,
maintaining the diet plan until a weight or other goal is achieved or indefinitely for maintenance of the achieved or desired weight.

2. A method of modulating the weight of an individual comprising:
a) calculating a base metabolic rate of the individual;
b) assigning an equation for calculating a daily caloric intake dependent on the base metabolic rate to each of a plurality of categories;
c) generating a pseudorandom sequence of the categories over a block of days, wherein the sequence is not known to the individual whose weight is being modulated;
d) calculating the daily caloric intake of for a particular day within the block of days;
g) revealing to the individual the daily caloric value of the particular day no earlier than 24 hours prior to the particular day
h) repeating steps d and g for the entire block of days.
